(19) United States
(12) Patent Application Publication
(10) Pub. No.: US 2024/0298958 A1
(43) Pub. Date: Sep. 12, 2024

(54) LARYNGEAL ELEVATION MEASUREMENT DEVICE

(71) Applicant: Murata Manufacturing Co., Ltd., Nagaokakyo-shi (JP)

(72) Inventors: Kenta SUZUKI, Nagaokakyo-shi (JP); Takayoshi OBATA, Nagaokakyo-shi (JP); Tomoki OUCHI, Nagaokakyo-shi (JP); Koji TANAKA, Nagaokakyo-shi (JP)

(21) Appl. No.: 18/591,575

(22) Filed: Feb. 29, 2024

(30) Foreign Application Priority Data
Mar. 7, 2023 (JP) 2023-034794

(51) Int. Cl. A61B 5/00; A61B 5/11

(52) U.S. Cl. CPC A61B 5/4205; A61B 5/1107; A61B 5/6822; A61B 5/7282; A61B 5/742; A61B 5/746

(57) ABSTRACT

A laryngeal elevation measurement device is provided that includes first and second displacement sensors and a control unit. The first displacement sensor is attachable to a neck of a test subject. The second displacement sensor 22 attachable to a site that is part of the neck of the test subject and that is located at a predetermined distance from the first displacement sensor. The control unit acquires the displacement amounts of the first and second displacement sensors. The control unit estimates positional changes of the laryngeal prominence of the test subject based on changes over time in the displacement amount of the first displacement sensor and changes over time in the displacement amount of the second displacement sensor, and the predetermined distance. The control unit outputs a result of estimation.

### CROSS REFERENCE TO RELATED APPLICATIONS

This application claims priority to Japanese Patent Application No. 2023-034794 filed on Mar. 7, 2023, the entire contents of which are hereby incorporated herein by reference.

### TECHNICAL FIELD

The present invention relates to a laryngeal elevation measurement device.

### BACKGROUND

Japanese Patent Application Publication No. 2022-7091 discloses swallowing movement measurement device that includes a sensor in sheet form, a press, and a measuring instrument. The sensor is a capacitive sensor that determines capacitive changes by detecting movements of the thyroid cartilage of a test subject. The press is used to press part of the sensor against the body surface in the laryngeal part of the test subject. The swallowing movement measurement device measures swallowing movements of the test subject based on the capacitive changes determined by the sensor.

The sensor of the swallowing movement measurement device described therein not only detects movements of the larynx of the test subject in the process of swallowing, but it also determines capacitive changes associated with movements of the neck of the test subject. Accordingly, measurements taken by the sensor are also reflective of movements of the test subject other than swallowing movements of the test subject. This configuration makes it difficult to detect, with high accuracy, the swallowing movements excluding other movements when detection results obtained by the swallowing movement measurement device.

### SUMMARY OF THE INVENTION

In view of the aforementioned problems, the exemplary aspects provide a laryngeal elevation measurement device that includes a first displacement sensor, a second displacement sensor, and a control unit. The first displacement sensor is attachable to a neck of a test subject. The second displacement sensor is attachable to a site that is part of the neck of the test subject and that is located at a predetermined distance from the first displacement sensor. The control unit is configured to acquire a displacement amount of the first displacement sensor and a displacement amount of the second displacement sensor. The control unit is configured to estimate positional changes of a laryngeal prominence of the test subject based on changes over time in the displacement amount of the first displacement sensor, changes over time in the displacement amount of the second displacement sensor, and the predetermined distance. The control unit further outputs a result of estimation.

According to the above configuration, the positional changes of the laryngeal prominence are estimated based on changes over time in the displacement amount acquired from each of the displacement sensors. The user can grasp in detail the state of the laryngeal prominence of the test subject based on an output of the result of estimation of positional changes of the laryngeal prominence. Accordingly, swallowing movements can be detected with high accuracy.

The state of the laryngeal prominence of the test subject is grasped in detail and swallowing movements can be detected exclusively according to the exemplary aspects.

### BRIEF DESCRIPTION OF THE DRAWINGS

FIG. 1 is an overall view of a laryngeal elevation measurement device according to an exemplary aspect.

FIG. 2 illustrates a state in which a main body of the laryngeal elevation measurement device is attached to a test subject according to an exemplary aspect.

FIG. 3 is a flowchart of a procedure by which a control unit performs image generation control according to an exemplary aspect.

FIG. 4 is an example image generated by the image generation control according to an exemplary aspect.

FIG. 5 is a flowchart of a procedure by which the control unit performs swallowing movement estimation control according to an exemplary aspect.

FIG. 6 is a flowchart of a procedure by which the control unit performs a variation of the swallowing movement estimation control according to an exemplary aspect.

FIG. 7 is an overall view of a variation of the laryngeal elevation measurement device according to an exemplary aspect.

FIG. 8 illustrates a state in which a main body of the variation of the laryngeal elevation measurement device is attached to a test subject according to an exemplary aspect.

FIG. 9 is example images generated by a variation of the image generation control according to an exemplary aspect.

### DETAILED DESCRIPTION

Hereinafter, a laryngeal elevation measurement device according to an exemplary embodiment is described with reference to the accompanying drawings. It is noted that constituent elements illustrated in some of the accompanying drawings are scaled up for the purpose of facilitating the understanding of the description. Some constituent elements are not drawn to scale, and the scale ratio may vary from drawing to drawing as should be appreciated to those skilled in the art.

### Overall Configuration

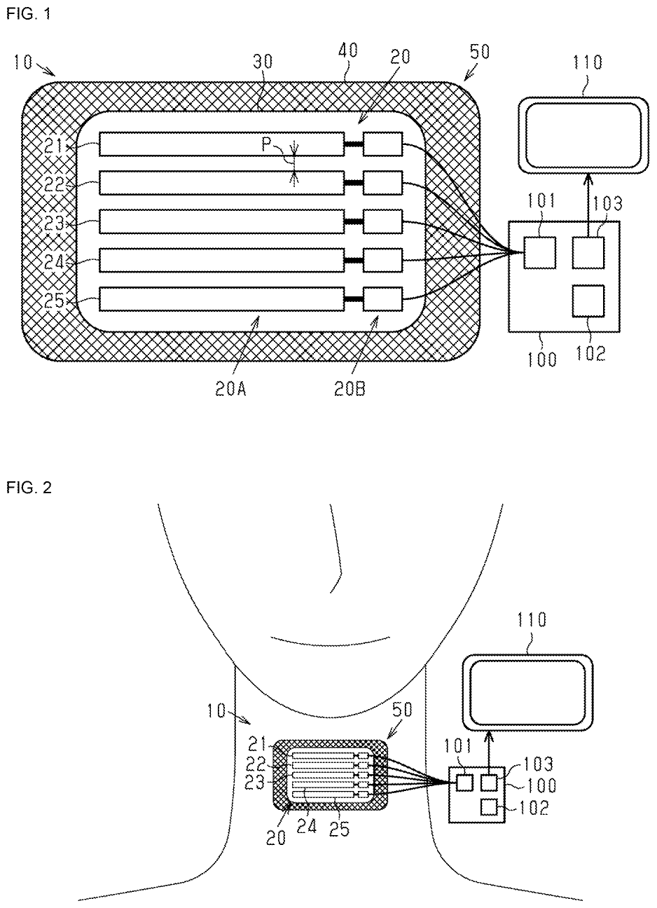

Referring to FIG. 1, a laryngeal elevation measurement device 10 includes a main body 50 and a controller 100. The main body 50 is configured to be attached to a test subject. The controller 100 is electrically connected to the main body 50. The main body 50 includes a sensor sheet 30, five displacement sensors 20, and a fastening member 40.

As shown, the sensor sheet 30 is substantially rectangular when viewed in plan. The sensor sheet 30 is made of elastic synthetic resin. The sensor sheet 30 is preferably made of a material of low elastic modulus, such as polyurethane, acrylic, or silicone resin.

The displacement sensors 20 are fitted to (e.g., coupled to) one main surface of the sensor sheet 30. Each of the displacement sensors 20 is, as a whole, strip-shaped. That is, the displacement sensors 20 each extend along the long sides of the sensor sheet 30. The five displacement sensors 20 are separated from each other in the direction of the short sides of the sensor sheet 30 and are arranged in parallel to each other. The five displacement sensors 20 are arranged at equal intervals in the exemplary aspect.

The displacement sensors 20 each include a sensor part 20A and a detection part 20B. The sensor part 20A is strip-shaped. The sensor part 20A is parallel to the long sides of the sensor sheet 30. The sensor part 20A is made of an electrical conductor and can be made of a material whose resistance value to expansion and contraction changes considerably. For example, the sensor part 20A is made of a mixture of metal powder (e.g., silver powder or copper powder) and elastomer-based resin (e.g., silicone).

The sensor part 20A is elastically deformable. As the length of the long sides of the sensor part 20A changes due to elastic deformation, the resistance value of the sensor part 20A changes. For example, the displacement sensors 20 are each in the form of a straight line when not being subjected to external force. The displacement sensors 20 curve due to the application of external force, and the resistance value of the displacement sensors 20 increases.

As further shown, the detection part 20B is electrically connected to the sensor part 20A. The detection part 20B is configured to convert changes in the resistance value of the sensor part 20A into the displacement amount. That is, the detection part 20B determines the displacement amount of the sensor part 20A at the time of deformation from a straight shape to a curved shape. The detection part 20B then transmits a signal indicative of the displacement amount.

According to the exemplary aspect, the fastening member 40 is in the form of a sheet. Examples of the material of the fastening member 40 include polyurethane rubber, silicone rubber/nitrile rubber sponge, chloroprene rubber sponge, and ethylene rubber sponge. The fastening member 40 is disposed on the opposite side from the displacement sensors 20 with the sensor sheet 30 being located between the fastening member 40 and the displacement sensors 20. The fastening member 40 is bonded to the sensor sheet 30 and preferably has a size larger than the sensor sheet 30. The fastening member 40 covers substantially the entirety of the sensor sheet 30. Moreover, the fastening member 40 has an adhesive surface on the opposite side from the sensor sheet 30. The main body 50 can be configured to be attached to a test subject with the fastening member 40 stuck on the test subject.

The five displacement sensors 20 are herein referred to as first to fifth displacement sensors and denoted by 21 to 25, respectively, as illustrated in FIG. 1. Referring to FIG. 2, the five displacement sensors 20 are attached to the neck of the test subject. With the five displacement sensors 20 being arranged along the cervical vertebrae of the test subject, the uppermost displacement sensor is the first displacement sensor 21. In the present embodiment, the first displacement sensor 21 is located at a level higher than the laryngeal prominence of the test subject under normal conditions, that is, in a state in which the test subject is not making swallowing movements. The second displacement sensor 22 is located below the first displacement sensor 21 and is attached to a site located at a predetermined distance P from the first displacement sensor 21. The third displacement sensor 23, the fourth displacement sensor 24, and the fifth displacement sensor 25 are located in this order below the second displacement sensor 22 and are arranged with the predetermined distance P therebetween. In the state in which the test subject is not making swallowing movements, the laryngeal prominence of the test subject is in positional agreement with any one of the second displacement sensor 22 to the fifth displacement sensor 25. In operation, when the test subject makes swallowing movements, the laryngeal prominence of the test subject moves upward and comes close to the first displacement sensor 21. That is, the first displacement sensor 21 is placed in the uppermost position within the range of motion of the laryngeal prominence associated with swallowing movements of the test subject.

### Controller

Referring to FIG. 1, the controller 100 includes a control unit 101, a storage unit 102, and a message unit 103.

The control unit 101 is connected to the respective displacement sensors 20 via codes. The control unit 101 receives signals transmitted by the displacement sensors 20. That is, the control unit 101 acquires the displacement amount from each of the displacement sensors 20. Upon acquisition of the displacement amount from each of the displacement sensors 20, the control unit 101 stores the displacement amount in the storage unit 102 in connection with the time of acquisition of the displacement amount. The control unit 101 is then configured to estimate the position of the laryngeal prominence of the test subject based on changes over time in the displacement amount of each of the displacement sensors 20. The control unit 101 then outputs a result of estimation to a display 110 via the message unit 103.

The control unit 101 contains circuitry including one or more processors. For example, the control unit 101 can be circuitry including at least one dedicated hardware circuit, such as an application-specific integrated circuit (ASIC), or may be circuitry including dedicated hardware circuits for use in combination.

The storage unit 102 is a storage medium that is readable by the control unit 101. Various kinds of processing to be performed by the control unit 101 are stored as program data (e.g., computer instructions and/or code) in the storage unit 102. Control values that are necessary for the various kinds of processing to be performed by the control unit 101 are stored in advance in the storage unit 102. As mentioned above, the displacement amount of each of the displacement sensors 20 is stored in the storage unit 102 in connection with the time of acquisition of displacement amount, based on the processing performed by the control unit 101.

The message unit 103 is configured to conduct wired and/or wireless communication with the display 110. As mentioned above, the message unit 103 causes the display 110 to display the result of estimation in accordance with various kinds of processing performed by the control unit 101. The controller 100 includes peripheral circuits (not illustrated), such as a power supply circuit and a clock circuit.

### Image Generation Control

The control unit 101 follows a series of steps for image generation control by executing an image generation program stored in the storage unit 102. The control unit 101 starts image generation control when the controller 100 is turned on. The control unit 101 repeats the image generation control in a predetermined period until the controller 100 is turned off.

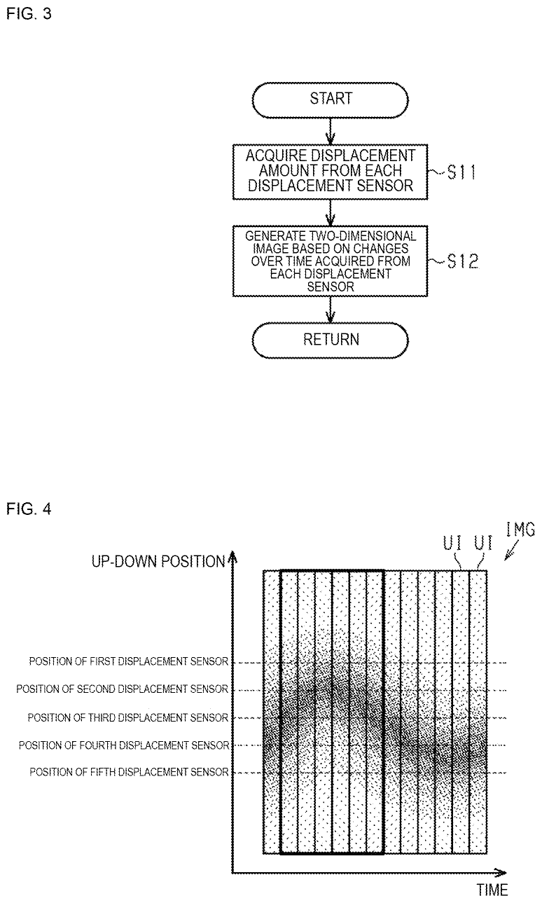

Referring to FIG. 3, the control unit 101 is configured to perform the processing in Step S11 at the start of the image generation control. In Step S11, the control unit 101 acquires the displacement amount from each of the displacement sensors 20. Upon acquisition of the displacement amount from each of the displacement sensors 20, the control unit 101 stores the displacement amount in the storage unit 102 in connection with the time of acquisition of the displacement amount. That is, the control unit 101 stores, in the storage unit 102, changes over time in the displacement amount of each of the displacement sensors 20. Subsequently, the control unit 101 performs the processing in Step S12.

In Step S12, the control unit 101 is configured to estimate the positional changes of the laryngeal prominence, based on the changes over time in the displacement amount acquired from each of the displacement sensors 20 and the predetermined distance P. More specifically, the position of the laryngeal prominence in the direction of the arrangement of the cervical vertebrae of the test subject is estimated as an up-down position of the laryngeal prominence by the control unit 101. The position of the top of the laryngeal prominence in the front-rear direction of the test subject is estimated as a front-rear position of the laryngeal prominence by the control unit 101. As a result of estimating the up-down position and the front-rear position of the laryngeal prominence, the control unit 101 is configured to generate a unit image UI. For example, the unit image UI is strip-shaped as illustrated in FIG. 4. The thickness of the black color in the unit image UI is reflective of the magnitude of displacement. The positions of the first displacement sensor 21 to the fifth displacement sensor 25 are indicated as positions in the longitudinal direction of the unit image UI. More specifically, the midsection in the longitudinal direction of the unit image UI in FIG. 4 corresponds to the position of the third displacement sensor 23. The second displacement sensor 22 and the first displacement sensor 21 are arranged on the upper side in this order, from closest to the midsection in the longitudinal direction of the unit image UI. The fourth displacement sensor 24 and the fifth displacement sensor 25 are arranged on the lower side in this order, from closest to the midsection in the longitudinal direction of the unit image UI. According to the exemplary aspect, the spot having the thickest black color in each unit image UI indicates the estimated up-down position of the laryngeal prominence. The thickness of the black color in the spot having the thickest black color in each unit image UI indicates the estimated result of the front-rear position of the laryngeal prominence.

When newly generating a unit image as described above, the control unit 101 is configured to combine the unit image UI and the previous unit images UI to newly generate a two-dimensional image IMG. More specifically, the control unit 101 arranges multiple unit images UI generated within a certain time period in chronological order to generate one two-dimensional image IMG. The certain time period is, for example, a few seconds. The two-dimensional image IMG illustrated in FIG. 4 is an image based on a Cartesian coordinate system that is defined by the horizontal axis representing time and the vertical axis representing the up-down position of the laryngeal prominence. As mentioned above, each unit image UI represents the up-down position and the front-rear position of the laryngeal prominence. The two-dimensional image IMG therefore represents changes over time in the up-down position of the laryngeal prominence and changes over time in the front-rear position of the laryngeal prominence.

According to an exemplary aspect, every time the control unit 101 newly generates a two-dimensional image IMG as described above, the control unit 101 also stores the two-dimensional image IMG in the storage unit 102. At this time, the control unit 101 deletes the previous two-dimensional image IMG from the storage unit 102. That is, the control unit 101 is configured to update the storage unit 102 by replacing the previous two-dimensional image IMG with the latest two-dimensional image IMG. In this way, the control unit 101 completes a series of steps for image generation control. The control unit 101 then starts image generation control again.

### Swallowing Movement Estimation Control

The control unit 101 follows a series of steps for swallowing movement estimation control by executing a swallowing movement estimation control program stored in the storage unit 102. The control unit 101 performs the swallowing movement estimation control only once when the laryngeal elevation measurement device 10 is turned on.

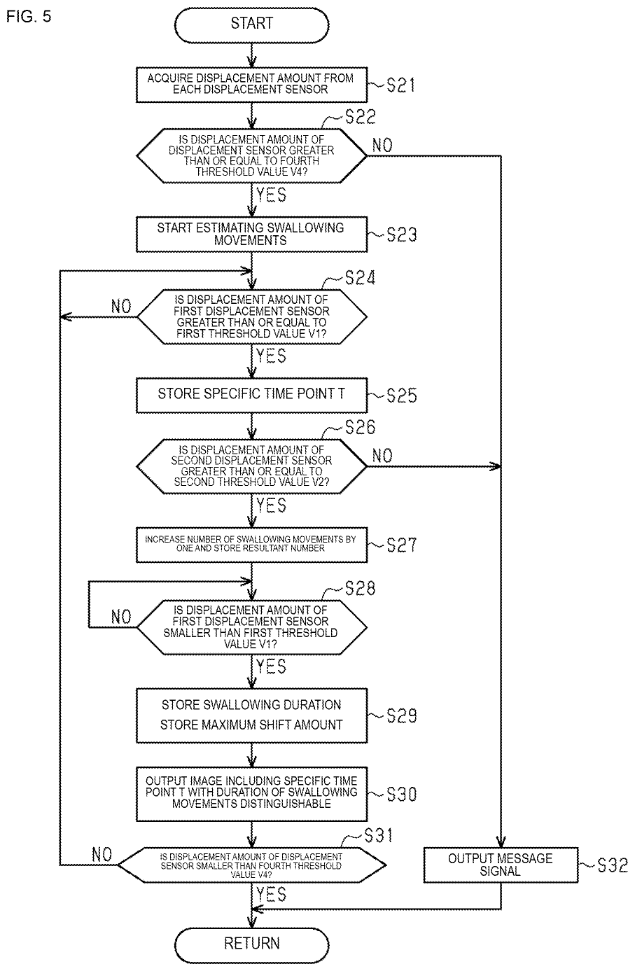

Referring to FIG. 5, the control unit 101 performs the processing in Step S21 at the start of the swallowing movement estimation control. In Step S21, the control unit 101 is configured to acquire the displacement amount from each of the displacement sensors 20. Subsequently, the control unit 101 performs the processing in Step S22.

In Step S22, the control unit 101 determines whether the displacement amount of each of the displacement sensors 20 is greater than or equal to a predetermined fourth threshold value V4. The fourth threshold value V4 is predetermined through, for example, tests or simulation and is a slightly smaller value than the minimum value of the displacement amount that is expected to be determined by the displacement sensors 20 when being attached to the neck of the test subject. When the displacement amount of every one of the displacement sensors 20 is greater than or equal to the fourth threshold value V4, the control unit 101 determines YES in Step S22. When the displacement amount of at least one of the displacement sensors 20 is smaller than the fourth threshold value V4, the control unit 101 determines NO in Step S22. When determining NO in Step S22, the control unit 101 proceeds to Step S32.

In Step S32, the control unit 101 outputs, to the display 110 via the message unit 103, a message signal for causing the display 110 to display a message for the user. Upon receipt of the message signal, the display 110 is further configured to display a message for alerting the user that the laryngeal elevation measurement device 10 is unable to properly estimate the position of the laryngeal prominence of the test subject. When the displacement amount of every one of the displacement sensors 20 is smaller than the fourth threshold value V4, the control unit 101 does not start estimating the positional changes of the laryngeal prominence. When the displacement amount of at least one of the displacement sensors 20 is smaller than the fourth threshold value V4, the control unit 101 is configured to alert the user that the position of the laryngeal prominence of the test subject is unable to be estimated. The laryngeal elevation measurement device 10 is unable to properly estimate the position of the laryngeal prominence of the test subject, for example, when the main body 50 is not attached to the test subject or when any of the displacement sensors 20 has abnormality. The control unit 101 then ends a series of steps for the swallowing movement estimation control and powers off the laryngeal elevation measurement device 10.

When determining YES in Step S22, the control unit 101 proceeds to Step S23. In Step S23, the control unit 101 starts estimating swallowing movements based on the positional changes of the laryngeal prominence of the test subject. Subsequently, the control unit 101 performs the processing in Step S24.

In Step S24, the control unit 101 is configured to acquire the displacement amount from each of the displacement sensors 20. The control unit 101 then determines whether the displacement amount acquired from the first displacement sensor 21 is greater than or equal to a first threshold value V1. The first threshold value V1 is predetermined through, for example, tests or simulation and is a slightly smaller value than the displacement amount that is expected to be determined by the first displacement sensor 21 at the time when the test subject makes swallowing movements. When determining NO in Step S24, the control unit 101 returns to Step S24. That is, the control unit 101 repeats the processing in Step S24 until the displacement amount of the first displacement sensor 21 reaches or exceeds the first threshold value V1. When determining YES in Step S24, the control unit 101 proceeds to Step S25.

In Step S25, the control unit 101 is configured to store a specific time point T in the storage unit 102. The specific time point T is the point in time when the displacement amount of the first displacement sensor 21 becomes greater than or equal to the first threshold value V1. In other words, the specific time point T is set by the control unit 101 as the point in time when the displacement amount of the first displacement sensor 21 below the first threshold value V1 reaches or exceeds the first threshold value V1. Subsequently, the control unit 101 performs the processing in Step S26.

In Step S26, the control unit 101 is configured to determine whether the displacement amount of the second displacement sensor 22 at the specific time point T is greater than or equal to a predetermined second threshold value V2. The second threshold value V2 is a preset value smaller than the first threshold value V1 and greater than or equal to the fourth threshold value V4. The second threshold value V2 is predetermined through, for example, tests or simulation as the lower limit of the value that is considered to be appropriate as the displacement amount of the second displacement sensor 22 adjacent to the first displacement sensor 21 at the time when the test subject makes swallowing movements. When determining NO in Step S26, the control unit 101 proceeds to Step S32 described above. When determining that the displacement amount of the second displacement sensor 22 at the specific time point T is below the second threshold value V2, the control unit 101 does not output the result of estimation. The control unit 101 then ends a series of steps for the swallowing movement estimation control and powers off the laryngeal elevation measurement device 10.

When determining YES in Step S26, the control unit 101 proceeds to Step S27. In Step S27, the control unit 101 is configured to increase the number of swallowing movements stored in the storage unit 102 by one. That is, the control unit 101 counts the number of swallowing movements of the test subject in the period during which the control unit 101 estimates the position of the laryngeal prominence. The count (i.e., the number of swallowing movements stored in the storage unit 102) is reset every time the swallowing movement estimation control is performed. Subsequently, the control unit 101 performs the processing in Step S28.

In Step S28, the control unit 101 is configured to acquire the displacement amount from each of the displacement sensors 20. The control unit 101 then determines whether the displacement amount of the first displacement sensor 21 is smaller than the first threshold value V1. When determining NO in Step S28, the control unit 101 repeats the processing in Step S28. That is, the control unit 101 repeats the processing in Step S28 until the displacement amount of the first displacement sensor 21 falls below the first threshold value V1. When determining YES in Step S28, the control unit 101 proceeds to Step S29.

In Step S29, the control unit 101 is configured to perform calculations to determine the swallowing duration that is the time taken for the test subject to make a single swallowing movement. More specifically, the swallowing duration is calculated by the control unit 101 as the length of time from the specific time point T to when the control unit 101 starts performing the processing in Step S29. The control unit 101 then calculates the maximum shift distance of the laryngeal prominence in a single swallowing movement. More specifically, the control unit 101 performs calculations to determine the distance between the lowermost position and the uppermost position where the displacement amount is at its maximum at each point in time within the swallowing duration. The control unit 101 stores the swallowing duration and the maximum shift distance in the storage unit 102 in connection with the number of swallowing movements. Subsequently, the control unit 101 performs the processing in Step S30.

In Step S30, the control unit 101 outputs a result of estimation concerning a first period including the point in time when the displacement amount of the first displacement sensor 21 below the first threshold value V1 reaches or exceeds the first threshold value V1. More specifically, the control unit 101 outputs, to the display 110 via the message unit 103, a two-dimensional image IMG associated with the first period including the specific time point T. The first period is a certain time period including a period before and after the specific time point T. The certain time period is, for example, less than one second. More specifically, the control unit 101 goes on standby at the specific time point T and waits until the certain time period elapses. The control unit 101 then outputs the two-dimensional image IMG generated by the image generation control as a result of estimation of positional changes of the laryngeal prominence. As mentioned above, the positional changes of the laryngeal prominence for a duration of a several seconds is represented as the two-dimensional image IMG. Accordingly, the two-dimensional image IMG associated with the first period includes positional changes of the laryngeal prominence in the certain time period including a period before and after the specific time point T. In the two-dimensional image IMG generated by the control unit 101, the time period during which the test subject is making swallowing movements (i.e., the swallowing duration) is made distinguishable from the time period over which the test subject is not making swallowing movements. For example, the time period during which the test subject is making swallowing movements is represented as a region enclosed in a rectangular frame (see FIG. 4) in the two-dimensional image IMG generated by the control unit 101. The control unit 101 causes the display 110 to display the swallowing duration and the maximum shift distance stored in Step S29 as numerical values in conformance with the two-dimensional image IMG displayed. Subsequently, the control unit 101 performs the processing in Step S31, as illustrated in FIG. 5.

In Step S31, the control unit 101 is configured to determine whether the displacement amount of each of the displacement sensors 20 is smaller than the fourth threshold value V4. When the displacement amount of at least one of the displacement sensors 20 is smaller than the fourth threshold value V4, the control unit 101 determines YES in Step S31. When the displacement amount of every one of the displacement sensors 20 is greater than or equal to the fourth threshold value V4, the control unit 101 determines NO in Step S31. When determining NO in Step S31, the control unit 101 returns to Step S24. In other words, the control unit 101 determines that the displacement sensors 20 are attached to the neck of the test subject, and the control unit 101 thus starts estimating the following swallowing movements. When determining YES in Step S31, the control unit 101 instructs the message unit 103 to cause the display 110 to display the latest number of swallowing movements stored in the storage unit 102 at that point in time. The control unit 101 then ends a series of steps for the swallowing movement estimation control.

### Actions Involved in Present Embodiment

The five displacement sensors 20 are configured to determine the displacement amount at their respective positions on the neck. That is, the control unit 101 is configured to acquire the displacement amount of each of the five sites of the neck at a certain point in time. The control unit 101 then approximates five numerical values of displacement with an appropriate function by taking the distance between the displacement sensors 20 into account. Accordingly, the position and the displacement amount of the laryngeal prominence at the time concerned can be determined by calculation. The control unit 101 generates a unit image UI that is reflective of the position and the displacement amount of the laryngeal prominence determined by calculation. The unit image UI is strip-shaped and is to be included in a two-dimensional image IMG.

At about the specific time point T, the control unit 101 is configured to determine that the test subject has made swallowing movements. The specific time point T is the point in time when the displacement amount of the first displacement sensor 21 below the first threshold value V1 reaches or exceeds the first threshold value V1. The laryngeal prominence of the test subject moves upward at the beginning of swallowing. This means that the laryngeal prominence of the test subject is on the move toward the first displacement sensor 21 during the first period. In operation, it is highly probable that the displacement amount of the second displacement sensor 22 adjacent to the first displacement sensor 21 reaches or exceeds the second threshold value V2 within the first period. Conversely, if the displacement amount of the second displacement sensor 22 in the first period is smaller than the second threshold value V2, there is a possibility that an error has occurred in the second displacement sensor 22.

### Effects of Present Embodiment

(1) In the embodiment described above, the estimation of positional changes of the laryngeal prominence is based on changes over time in the displacement amount acquired from each of the displacement sensors 20. The user can grasp or understand in detail the state of the laryngeal prominence of the test subject based on an output of the result of estimation of positional changes of the laryngeal prominence. Accordingly, swallowing movements can be detected with high accuracy.
(2) In the embodiment described above, a two-dimensional image IMG associated with the first period including the specific time point T is output provided that the displacement amount of the first displacement sensor 21 below the first threshold value V1 has reached or exceeded the first threshold value V1. That is, in the embodiment described above, a two-dimensional image IMG associated with the first period is output in which the test subject is highly likely to have performed swallowing movements. The user can thus acquire a two-dimensional image IMG that is highly likely to reflect the swallowing movements made in the period.
(3) In the embodiment described above, the control unit 101 is configured to output a two-dimensional image IMG associated with the certain time period including the specific time point T provided that the displacement amount of the second displacement sensor 22 at the specific time point T is greater than or equal to the second threshold value V2. If the displacement amount of the second displacement sensor 22 at the specific time point T is greater than or equal to the second threshold value V2, the laryngeal elevation measurement device 10 is probably detecting swallowing movements correctly. Thus, a two-dimensional image IMG at a time when swallowing movements are not correctly detected is prevented from being output.
(4) In the embodiment described above, the control unit 101 is configured to output a message signal provided that the displacement amount of the second displacement sensor 22 at the specific time point T is smaller than the second threshold value V2. If the displacement amount of the second displacement sensor 22 at the specific time point T is smaller than the second threshold value V2, the second displacement sensor 22 is probably not working properly. That is, according to this configuration, the user can understand that the second displacement sensor 22 is possibly not working properly.
(5) In the embodiment described above, the control unit 101 is configured to start estimating the positional changes of the laryngeal prominence provided that the displacement amount of every one of the displacement sensors 20 is greater than or equal to the fourth threshold value V4. In this aspect, it is highly probable that the displacement sensors 20 are elastically deformed along the surface of the neck of the test subject when the displacement amount of every one of the displacement sensors 20 is greater than or equal to the fourth threshold value V4. Thus, according to this configuration, swallowing movements can be estimated with the displacement sensors 20 being attached properly.
(6) In the embodiment described above, the control unit 101 is configured to output a message signal to alert the user provided that the displacement amount of at least one of the displacement sensors 20 is smaller than the fourth threshold value V4. It may be determined that the displacement sensors 20 are not attached properly when the displacement amount of at least one of the displacement sensors 20 is smaller than the fourth threshold value V4. According to this configuration, the user can understand that the displacement sensors 20 are not attached properly.
(7) In the embodiment described above, the control unit 101 is configured to output the result of estimation in the form of a two-dimensional image IMG based on a Cartesian coordinate system defined by the horizontal axis representing time and the vertical axis representing the up-down position. According to this configuration, the user can thus intuitively recognize the positional changes of the laryngeal prominence by using images.
(8) In the embodiment described above, the two-dimensional image IMG is an image in which the front-rear position of the laryngeal prominence is expressed as chromatic changes. The use of chromatic changes enables the two-dimensional image IMG to vicariously express three-dimensional information.
(9) In the embodiment described above, the control unit 101 is configured to display the two-dimensional image IMG in which the time period over which the test subject is making swallowing movements is enclosed in a rectangular frame in the two-dimensional image IMG. That is, the control unit 101 displays the time period over which the test subject is making swallowing movements distinguishable from the time period over which the test subject is not making swallowing movements. According to this configuration, it is easier to check positional changes of the laryngeal prominence during the time period over which the test subject is making swallowing movements.

### Variations

The embodiment described above and the following variations may be combined as long as there is no technical contradiction between them.

In the embodiment described above, the configuration of the main body 50 is not limited to the example according to the embodiment. For example, the main body 50 may include two or more sensor sheets 30, and each of the displacement sensors 20 may be bonded to the corresponding one of the sensor sheets 30. Alternatively, the sensor sheet(s) 30 and the fastening member 40 of the main body 50 may be eliminated, and the displacement sensors 20 may be attached to the neck with an adhesive tape or the like.

In the embodiment described above, at least two displacement sensors 20 are provided. In other words, it suffices that the first displacement sensor 21 and the second displacement sensor 22 be included in the laryngeal elevation measurement device 10. If at least two displacement sensors 20 are provided, positional changes of the laryngeal prominence of the test subject can be estimated based on the positional relationship between the first displacement sensor 21 and the second displacement sensor 22.

In the embodiment described above, the sensor part 20A is strip-shaped. Alternatively, the sensor part 20A may be in the form of a line, for example.

It is noted that in the embodiment described above, the specific configuration of the displacement sensors 20 is not limited to the example according to the embodiment as long as the displacement sensors 20 are capable of measuring the displacement amount. For example, the displacement sensors 20 each can be a capacitive sensor that includes a pair of electrodes and a dielectric layer disposed between the electrodes and that is configured to detect capacitive changes to determine the degree of distortion. Alternatively, the displacement sensors 20 each can be configured to determine the degree of distortion by using light, a camera, or the like. Alternatively, the displacement sensors 20 each can be made of a piezoelectric film.

In an exemplary aspect, the control unit 101 can skip the swallowing movement estimation control. In that case, every time the control unit 101 generates a two-dimensional image IMG by the image generation control, the control unit 101 may output the two-dimensional image IMG to the display 110. In this case, the display 110 displays two-dimensional images IMG changing over time, that is, moving images.

Moreover, the control unit 101 can skip the processing in Step S24 of the swallowing movement estimation control. In other words, the control unit 101 may set the specific time point T irrespective of whether the displacement amount of the first displacement sensor 21 is greater than or equal to the first threshold value V1 or less than the first threshold value V1. In this case, for example, the user may input the specific time point T in correspondence with the occurrence of swallowing movements of the test subject.

Furthermore, the control unit 101 can skip the processing in each of Steps S22 and S31 of the swallowing movement estimation control. In that case, the control unit 101 may end the swallowing movement estimation control upon the lapse of a period predetermined by, for example, the user. When skipping the processing in Step S22, the control unit 101 may also skip the processing in Step S32.

It is noted that the message signal output by the control unit 101 in Step S32 of the swallowing movement estimation control is not limited to a signal in a particular form. Moreover, the destination of output of the message signal is not limited to the display 110. Any message signal and any recipient that receives the message signal may be used as long as the message signal is configured to perform notification that the laryngeal elevation measurement device 10 is not in a state appropriate for measurement of swallowing movements.

The control unit 101 may skip the processing in Step S27 of the swallowing movement estimation control. That is, the control unit 101 may skip the step of counting the number of swallowing movements of the test subject.

Moreover, the control unit 101 may skip the calculation of the swallowing duration or the calculation of the maximum shift distance of the laryngeal prominence in Step S29 of the swallowing movement estimation control. Furthermore, the control unit 101 may skip the processing in Step S29 of the swallowing movement estimation control. That is, the control unit 101 may skip both the calculation of the swallowing duration and the calculation of the maximum shift distance of the laryngeal prominence.

If there is no need to estimate the time of completion of the swallowing movements, the control unit 101 may skip the processing in Step S28 of the swallowing movement estimation control.

In the two-dimensional image IMG displayed by the display 110 in Step S30 of the swallowing movement estimation control, the front-rear position may be represented by the vertical axis, and the up-down position may be expressed as chromatic changes. In this case, the chromatic changes include changes in hue, changes in brightness, changes in saturation, or the like.

The control unit 101 can be configured to display the two-dimensional image IMG in Step S30 of the swallowing movement estimation control, where the time period over which the test subject is making swallowing movements may be made distinguishable from the time period over which the test subject is not making swallowing movements, in a method different from the one described in the embodiment described above. For example, the time period over which the test subject is making swallowing movements and the time period over which the test subject is not making swallowing movements may be displayed in different colors in the two-dimensional image. The control unit 101 may display the time period over which the test subject is making swallowing movements and the time period over which the test subject is not making swallowing movements not distinguishable from each other in the two-dimensional image.

According to an exemplary aspect, the control unit 101 generates the two-dimensional image in which at least positional changes of the laryngeal prominence are reflected. In this aspect, the two-dimensional image does not necessarily include large or small changes in the amount of displacement. In that case, the control unit 101 may, for example, estimate positional changes of the laryngeal prominence by plotting the positions where the displacement amount is at its maximum at the respective points in time.

In Step S26 of the swallowing movement estimation control, the control unit 101 compares the displacement amount of the second displacement sensor 22 with the second threshold value V2 to determine whether the second displacement sensor 22 is working properly. The control unit 101 can be configured to perform processing different from Step S26 of the swallowing movement estimation control to determine whether the second displacement sensor 22 is working properly.

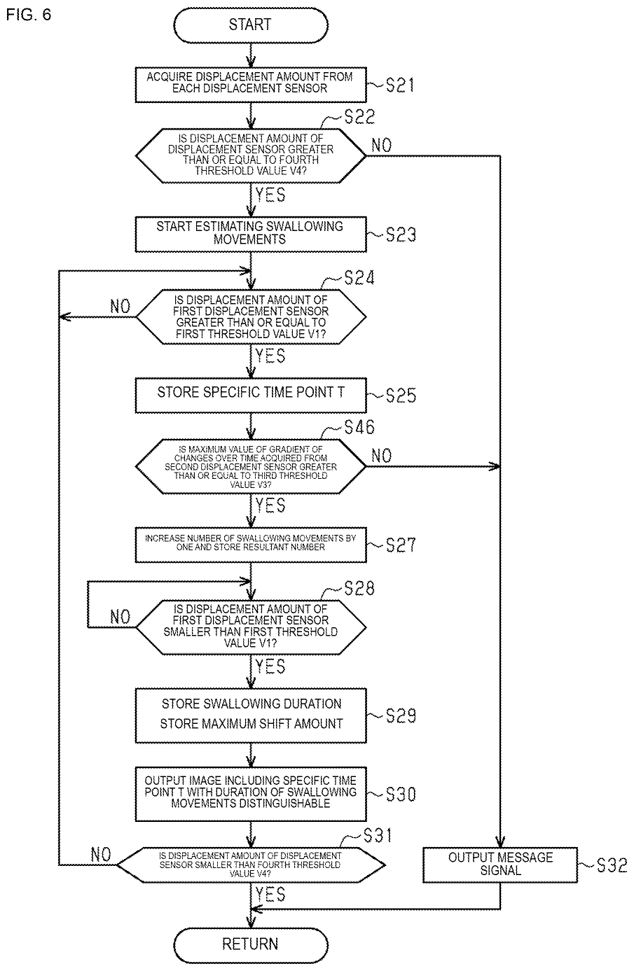

For example, in a variation of the swallowing movement estimation control illustrated in FIG. 6, the control unit 101 can be configured to perform the processing in Step S46 instead of performing the processing in Step S26 in the embodiment described above. In Step S46, the control unit 101 determines whether the maximum value of the gradient of changes over time in the displacement amount of the second displacement sensor 22 in the first period including the specific time point T is greater than or equal to a third threshold value V3. The control unit 101 determines the gradient of changes over time in the displacement amount of the second displacement sensor 22 by obtaining the displacement amount of the second displacement sensor 22 measured at a certain time point in the first period and the displacement amount of the second displacement sensor 22 measured at a time point before the certain time point by a unit period and by subtracting the latter from the former. According to the exemplary aspect, the third threshold value V3 is predetermined through, for example, tests or simulation as the maximum value of the gradient of the amount of displacement of the second displacement sensor 22 adjacent to the first displacement sensor 21 within the time period over which the test subject is making swallowing movements. When determining NO in Step S46, the control unit 101 proceeds to Step S32 of the embodiment described above. That is, when determining that the absolute value of the gradient of changes over time in the displacement amount of the second displacement sensor 22 in the first period is smaller than the third threshold value V3, the control unit 101 does not output the result of estimation. When determining that the absolute value of the gradient of changes over time in the displacement amount of the second displacement sensor 22 in the first period is smaller than the third threshold value V3, the control unit 101 outputs a message signal to alert the user that the positional changes of the laryngeal prominence of the test subject cannot be estimated. When determining YES in Step S46, the control unit 101 proceeds to Step S27 and subsequent steps in the embodiment described above. That is, the estimation result of positional changes of the laryngeal prominence within the certain time period including the specific time point T is output provided that the absolute value of the gradient of changes over time in the displacement amount of the second displacement sensor 22 at the specific time point T is greater than or equal to the third threshold value V3.

In the configuration described above, more than one kind of value may be regarded as the gradient of changes over time in the displacement amount. For example, the gradient of changes over time in the displacement amount may be a value obtained by differentiation. Alternatively, the gradient of changes over time in the displacement amount may be a value obtained based on a tangent line drawn in a graph of displacement amount and time. Alternatively, the gradient of changes over time in the displacement amount may be a value obtained based on the angle of inclination of a straight line drawn in a graph of displacement amount and time and connecting a point where the displacement amount of the first displacement sensor 21 has yet to exceed the first threshold value V1 to a subsequent point where the displacement amount of the first displacement sensor 21 is greater than the first threshold value V1.

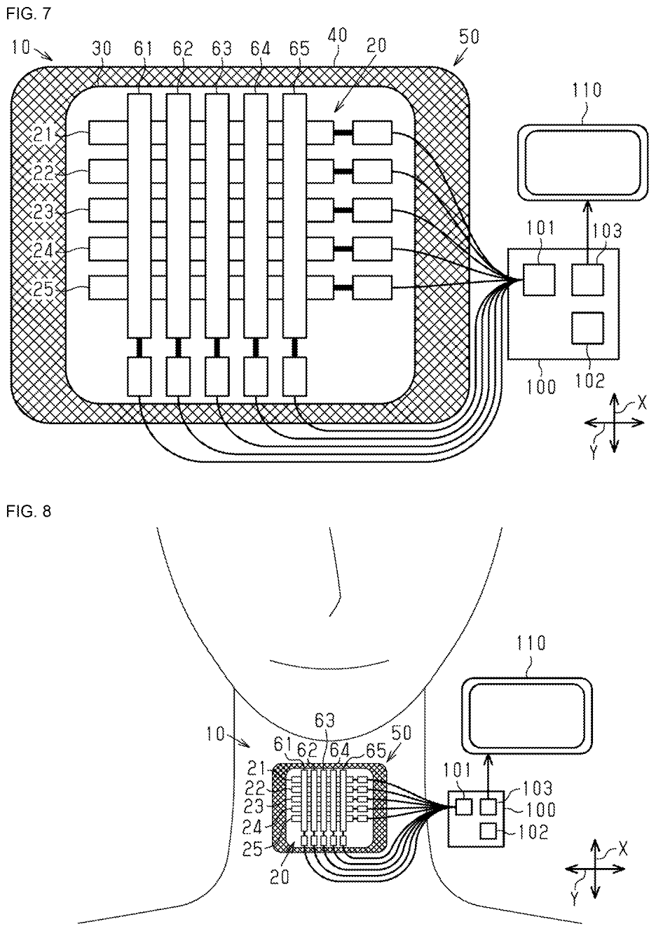

In the embodiment described above, the laryngeal elevation measurement device 10 includes displacement sensors 20 that are arranged perpendicularly to the arrangement of the first displacement sensor 21. Referring to FIG. 7, the displacement sensors 20 include a first lateral displacement sensor 61, a second lateral displacement sensor 62, a third lateral displacement sensor 63, a fourth lateral displacement sensor 64, and a fifth lateral displacement sensor 65 in addition to the first displacement sensor 21 to the fifth displacement sensor 25. The lateral displacement sensors are structurally identical to the first displacement sensor 21 and the other displacement sensors 20 mentioned in the embodiment described above.

The axis extending in the direction of the arrangement of the first displacement sensor 21 and the second displacement sensor 22 is herein referred to as a first axis X. The direction from the first displacement sensor 21 toward the second displacement sensor 22 is herein referred to as a first direction. The axis being orthogonal to the first axis X and extending along the sensor sheet 30 is herein referred to as a second axis Y. One of the directions along the second axis Y is herein referred to as a second direction. That is, the second direction intersects the first direction. According to the exemplary aspect, the first lateral displacement sensor 61 to the fifth lateral displacement sensor 65 are each in the form of a strip extending along the first axis X. The second lateral displacement sensor 62 is attached to a site that is located away from the first lateral displacement sensor 61 in the second direction. As illustrated in FIG. 8, specifically, the second lateral displacement sensor 62 is located on the right side of the first lateral displacement sensor 61 as viewed from the front of the test subject. On the right side of the second lateral displacement sensor 62, the third lateral displacement sensor 63, the fourth lateral displacement sensor 64, and then the fifth lateral displacement sensor 65 are located in this order at intervals. With the addition of the first lateral displacement sensor 61 to the fifth lateral displacement sensor 65, the control unit 101 can estimate the positional changes of the laryngeal prominence in the direction of the second axis Y two-dimensionally.

The control unit 101 in this exemplary aspect is configured to estimate the position of the laryngeal prominence of the test subject based on changes over time in the displacement amount of each of the first displacement sensor 21 to the fifth displacement sensor 25 and changes over time in the displacement amount of each of the first lateral displacement sensor 61 to the fifth lateral displacement sensors 65. More specifically, the control unit 101 is configured to estimate the position of the laryngeal prominence of the test subject as the position on an imaginary plane defined by the first axis X and the second axis Y orthogonal to each other, and outputs the result of estimation.

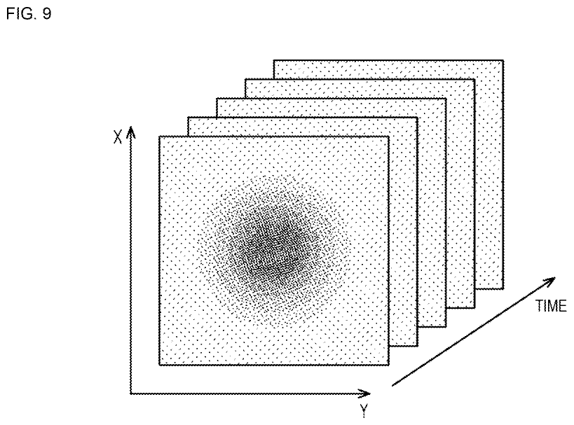

As illustrated in FIG. 9, the control unit 101 generates a two-dimensional image whose vertical axis is the first axis X and lateral axis is the second axis Y as a result of estimating the up-down position and the lateral position of the laryngeal prominence. The thickness of the black color in the image is reflective of the magnitude of displacement. The positions of the first displacement sensor 21 to the fifth displacement sensor 25 are indicated as positions in the direction of the first axis X. The positions of the first lateral displacement sensor 61 to the fifth lateral displacement sensor 65 are indicated as positions in the direction of the second axis Y. The control unit 101 generates two or more such images in every predetermined time period. The control unit 101 generates moving images by combining the images in the respective predetermined periods. The moving images represent changes over time in the position of the laryngeal prominence in the direction of the first axis X and changes over time in the position of the laryngeal prominence in the direction of the second axis Y. In the example illustrated in FIG. 7, it suffices that at least the first lateral displacement sensor 61 and the second lateral displacement sensor 62 be provided as the lateral displacement sensors.

In the embodiment described above, it is noted that each threshold value is not necessarily set in advance, and each threshold value may be calculated every time a measurement is conducted. For example, the first threshold value V1 may be obtained by multiplying a preset value by a coefficient based on the temperature and humidity on the day of measurement.

The following describes technical ideas derived from the embodiment and variations described above.

In an exemplary aspect, a laryngeal elevation measurement device is provided that includes a first displacement sensor attachable to a neck of a test subject; a second displacement sensor attachable to a site that is part of the neck of the test subject and that is located at a predetermined distance from the first displacement sensor; and a control unit configured to acquire a displacement amount of the first displacement sensor and a displacement amount of the second displacement sensor. In this aspect, the control unit is configured to estimate positional changes of a laryngeal prominence of the test subject based on changes over time in the displacement amount of the first displacement sensor, changes over time in the displacement amount of the second displacement sensor, and the predetermined distance, and output a result of estimation.

In another exemplary aspect, the control unit is further configured to output the result of estimation concerning a first period including a point in time when the displacement amount of the first displacement sensor below a first threshold value reaches or exceeds the first threshold value.

In another exemplary aspect, the control unit is further configured to output the result of estimation concerning the first period when a maximum value of a gradient of the changes over time in the displacement amount of the second displacement sensor in the first period is greater than or equal to a third threshold value. Moreover, the control unit is configured not to output the result of estimation concerning the first period when the maximum value is smaller than the third threshold value.

In another exemplary aspect, the control unit is further configured to alert a user that the laryngeal elevation measurement device is unable to estimate the positional changes of the laryngeal prominence of the test subject when determining that a maximum value of a gradient of changes over time in the displacement amount of the second displacement sensor in the first period is smaller than a third threshold value.

In another exemplary aspect, the control unit is further configured to output the result of estimation concerning the first period when the displacement amount of the second displacement sensor at a specific time point is greater than or equal to a second threshold value smaller than the first threshold value. In this aspect, the control unit is further configured not to output the result of estimation when determining that the displacement amount of the second displacement sensor at the specific time point is smaller than the second threshold value. Moreover, the specific time point is the point in time when the displacement amount of the first displacement sensor below the first threshold value reaches or exceeds the first threshold value.

In another exemplary aspect, the control unit is further configured to start estimating the positional changes of the laryngeal prominence when determining that the displacement amount of the first displacement sensor and the displacement amount of the second displacement sensor are each greater than or equal to a fourth threshold value smaller than the first threshold value. In this aspect, the control unit is further configured not to start estimating the positional changes of the laryngeal prominence when determining that the displacement amount of the first displacement sensor and/or the displacement amount of the second displacement sensor is smaller than the fourth threshold value.

In yet another exemplary aspect, the control unit is further configured to alert a user that the laryngeal elevation measurement device is unable to estimate the positional changes of the laryngeal prominence of the test subject when determining that the displacement amount of the first displacement sensor and/or the displacement amount of the second displacement sensor is smaller than a fourth threshold value smaller than the first threshold value.

In yet another exemplary aspect, the control unit is further configured to output the result of estimation in a form of a two-dimensional image that is based on a Cartesian coordinate system defined by a horizontal axis representing time and a vertical axis representing an up-down position or a front-rear position and that contains chromatic changes expressing one of the up-down position and the front-rear positions that is not represented by the vertical axis. In this aspect, the up-down position is a position of the laryngeal prominence in a direction of an arrangement of cervical vertebrae of the test subject. Moreover, the front-rear position is a position of a top of the laryngeal prominence in a front-rear direction of the test subject.

In another exemplary aspect, the control unit is configured to express, numerically in conformance with the form of the two-dimensional image, at least one selected from the group of the number of swallowing movements of the test subject in a period during which the control unit estimates the position of the laryngeal prominence, swallowing duration that is time taken for the test subject to make a single swallowing movement, and a maximum shift distance of the laryngeal prominence in the single swallowing movement.

In another exemplary aspect, the control unit is further configured to display the two-dimensional image in which a time period over which the test subject is making swallowing movements is made distinguishable from a time period over which the test subject is not making the swallowing movements.

In another exemplary aspect, the laryngeal elevation measurement device further includes a first lateral displacement sensor attachable to the neck of the test subject; and a second lateral displacement sensor attachable to a site that is part of the neck of the test subject and that is located away from the first lateral displacement sensor in a second direction intersecting a first direction from the first displacement sensor toward the second displacement sensor. In this aspect, the control unit is further configured to estimate the position of the laryngeal prominence based on the changes over time in the displacement amount of the first displacement sensor, the changes over time in the displacement amount of the second displacement sensor, changes over time in a displacement amount of the first lateral displacement sensor, and changes over time in a displacement amount of the second lateral displacement sensor, and output the result of estimation.

What is claimed:

1. A laryngeal elevation measurement device comprising:
a first displacement sensor configured to be attached to a neck of a test subject;
a second displacement sensor configured to be attached to a site that is part of the neck of the test subject and that is located at a predetermined distance from the first displacement sensor; and
a control unit configured to:
acquire a displacement amount of the first displacement sensor and a displacement amount of the second displacement sensor,
estimate positional changes of a laryngeal prominence of the test subject based on changes over time in the displacement amount of each of the first and second displacement sensors and the predetermined distance, and
output a result based on the estimated positional changes.

2. The laryngeal elevation measurement device according to claim 1, wherein the control unit is further configured to output the result concerning a first period including a point in time when the displacement amount of the first displacement sensor reaches or exceeds a first threshold value.

3. The laryngeal elevation measurement device according to claim 2, wherein the control unit is further configured to:
output the result concerning the first period when a maximum value of a gradient of the changes over time in the displacement amount of the second displacement sensor in the first period is greater than or equal to a third threshold value, and
not output the result concerning the first period when the maximum value is smaller than the third threshold value.

4. The laryngeal elevation measurement device according to claim 2, wherein the control unit is further configured to generate an alert that the laryngeal elevation measurement device is unable to estimate the positional changes of the laryngeal prominence of the test subject when determining that a maximum value of a gradient of the changes over time in the displacement amount of the second displacement sensor in the first period is smaller than a third threshold value.

5. The laryngeal elevation measurement device according to claim 2, wherein the control unit is further configured to:
output the result concerning the first period when the displacement amount of the second displacement sensor at a specific time point is greater than or equal to a second threshold value that is smaller than the first threshold value, and
not output the result when the displacement amount of the second displacement sensor at the specific time point is smaller than the second threshold value,
wherein the specific time point is the point in time when the displacement amount of the first displacement sensor reaches or exceeds the first threshold value.

6. The laryngeal elevation measurement device according to claim 2, wherein the control unit is further configured to:
start estimating the positional changes of the laryngeal prominence when the displacement amount of the first displacement sensor and the displacement amount of the second displacement sensor are each greater than or equal to a fourth threshold value that is smaller than the first threshold value, and
not start estimating the positional changes of the laryngeal prominence when the displacement amount of at least one of the first displacement sensor and the second displacement sensor is smaller than the fourth threshold value.

7. The laryngeal elevation measurement device according to claim 2, wherein the control unit is further configured to generate an alert that the laryngeal elevation measurement device is unable to estimate the positional changes of the laryngeal prominence of the test subject when the displacement amount of at least one of the first displacement sensor and the second displacement sensor is smaller than a fourth threshold value that is smaller than the first threshold value.

8. The laryngeal elevation measurement device according to claim 1, wherein the control unit is further configured to output the result in a form of a two-dimensional image that is based on a Cartesian coordinate system that is defined by a horizontal axis representing time and a vertical axis representing an up-down position or a front-rear position and that contains chromatic changes expressing one of the up-down position and the front-rear position that is not represented by the vertical axis.

9. The laryngeal elevation measurement device according to claim 8, wherein:
the up-down position is a position of the laryngeal prominence in a direction of an arrangement of cervical vertebrae of the test subject, and
the front-rear position is a position of a top of the laryngeal prominence in a front-rear direction of the test subject.

10. The laryngeal elevation measurement device according to claim 8, wherein the control unit is further configured to express, numerically in conformance with the form of the two-dimensional image, at least one of:
a number of swallowing movements of the test subject in a period during which the control unit estimates the position of the laryngeal prominence,
a swallowing duration that is time taken for the test subject to make a single swallowing movement, and
a maximum shift distance of the laryngeal prominence in the single swallowing movement.

11. The laryngeal elevation measurement device according to claim 8, wherein the control unit is further configured to display the two-dimensional image in which a time period over which the test subject is making swallowing movements is made distinguishable from a time period over which the test subject is not making the swallowing movements.

12. The laryngeal elevation measurement device according to claim 1, further comprising:
a first lateral displacement sensor configured to be attached to the neck of the test subject; and
a second lateral displacement sensor configured to be attached to another site that is part of the neck of the test subject and that is located away from the first lateral displacement sensor in a second direction that intersects a first direction from the first displacement sensor toward the second displacement sensor.

13. The laryngeal elevation measurement device according to claim 12, wherein the control unit is further configured to:
estimate the change in position of the laryngeal prominence based on the changes over time in the displacement amount of the first displacement sensor, the changes over time in the displacement amount of the second displacement sensor, changes over time in a displacement amount of the first lateral displacement sensor, and changes over time in a displacement amount of the second lateral displacement sensor, and
output the result based on the estimated changes in position.

14. The laryngeal elevation measurement device according to claim 1, wherein the first and second displacement sensors are strip-shaped that are separate from each other and extend in a direction parallel to each other.

15. The laryngeal elevation measurement device according to claim 1, wherein the control unit comprises one or more processors configured to execute program instructions on memory for acquiring the respective displacement amounts, estimating the respective positional changes of the laryngeal prominence of the test subject, and outputting the result.

16. A method for measuring laryngeal elevation, the method comprising:
acquiring, by a control unit, a displacement amount of a first displacement sensor attached to a neck of a test subject;
acquiring, by the control unit, a displacement amount of a second displacement sensor that is attached to a site that is part of the neck of the test subject and that is located at a predetermined distance from the first displacement sensor;
estimating, by the control unit, positional changes of a laryngeal prominence of the test subject based on changes over time in the displacement amount of each of the first and second displacement sensors and the predetermined distance; and
outputting a result based on the estimated positional changes.

17. The method according to claim 16, further comprising outputting, by the control unit, the result concerning a first period including a point in time when the displacement amount of the first displacement sensor reaches or exceeds a first threshold value.

18. The method according to claim 17, further comprising:
outputting the result concerning the first period when a maximum value of a gradient of the changes over time in the displacement amount of the second displacement sensor in the first period is greater than or equal to a third threshold value; and
not outputting the result concerning the first period when the maximum value is smaller than the third threshold value.

19. The method according to claim 17, further comprising generating an alert when the positional changes of the laryngeal prominence of the test subject cannot be estimated when determining that a maximum value of a gradient of the changes over time in the displacement amount of the second displacement sensor in the first period is smaller than a third threshold value.

20. The method according to claim 16, further comprising outputting the result in a form of a two-dimensional image that is based on a Cartesian coordinate system that is defined by a horizontal axis representing time and a vertical axis representing an up-down position or a front-rear position and that contains chromatic changes expressing one of the up-down position and the front-rear position that is not represented by the vertical axis.
